(19) United States
(12) Patent Application Publication
(10) Pub. No.: US 2025/0281369 A1
(43) Pub. Date: Sep. 11, 2025

(54) Calming Sunscreen Body Butter

(71) Applicant: Brianna Colette Sudduth, Fort Worth, TX (US)

(72) Inventor: Brianna Colette Sudduth, Fort Worth, TX (US)

(21) Appl. No.: 18/601,992

(22) Filed: Mar. 11, 2024

(51) Int. Cl. A61K 8/06; A61K 36/23; A61K 36/53; A61K 36/73; A61K 36/87; A61K 36/889; A61Q 17/00; A61Q 17/04; A61Q 19/00; A61Q 19/08

(52) U.S. Cl. CPC A61K 8/06; A61K 36/23; A61K 36/53; A61K 36/73; A61K 36/87; A61K 36/889; A61Q 17/005; A61Q 17/04; A61Q 19/007; A61Q 19/08; A61K 2800/5922; A61K 2800/75

(57) ABSTRACT

A topical composition comprising at least 5 active plant-based ingredients which each have at least one activity that include but are not limited to: moisturizes skin that lasts hours after application, reduces the appearance of scars, provides UV protection without constant reapplication, tightens most skin types, helps with acne prone and blemished skin, reduces redness and skin irritation, battles free radicals, removes make up. This combination also leads to anti-inflammatory, antianxiety, and antimicrobial results after a small to moderate amount is applied the skin that is exposed to the elements.

### BACKGROUND OF THE INVENTION

Dry skin is a challenge for many people that live in dry climates. Some drier regions requires moisturizer to be applied several times a day. Many sunscreens also encourage applications after a certain number of hours.

### SUMMARY OF THE INVENTION

The invention has many other benefits, but the main aspect that makes this invention different is that is made with all natural ingredients and only needs to be applied once a day. It is truly a vegan product that provides moisture for an extended period of time, provides protection from the sun, and has calming effects using only a few ingredients for those with sensitive skin.

### BRIEF DESCRIPTION OF DRAWINGS

FIG. 1 represents the proportion of each of the ingredients as they are added to the individual glass jars of various sizes.

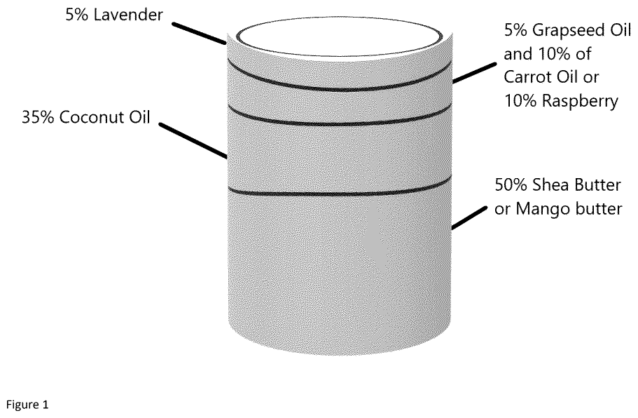

### DETAILED DESCRIPTION OF THE INVENTION

The invention includes the combination of the ingredients listed below. For those who are allergic to latex, mango butter is used instead of shea butter.
combination 1 Shea Butter: vitamins A and E (tocopherols), allantoin, phenolic compounds (catechins and quercetin), sterols, and triterpenes;
combination 1 Coconut Oil: lauric acid (41.8 grams per 100 grams of total composition), while other significant saturated fats are myristic acid (16.7 g), palmitic acid (8.6 g), and caprylic acid (6.8 g). Monounsaturated fats are 6% of total composition, and polyunsaturated fats are 2% (table);
combination 1 Lavender Oil: Linalool: 20-35%, Linalyl acetate: 30-55% Terpinen-4-ol: Moderate levels, Lavandulol: Moderate levels, 1,8-cineole: Variable levels, Camphor: Variable levels, Limonene: In small amounts, Tannins: In small amounts;
combination 1 Grapeseed Oil: Polyunsaturated fatty acids, Omega-6 fatty acids, Linoleic acid, Vitamin E, Phenolic antioxidants, Tocopherol, Linolenic acid, Quercetin, Resveratrol, Procyanidins, Carotenoids, Phytosterols
combination 1 Carrot Oil: Vitamin A, C, and E, Beta-carotene, Carotene, Antioxidants, Carotol, Daucene, Beta-Bisabolene, Daucol, Alpha-pinene, Sabinene, Beta caryophyllene
Combination 2 Raspberry Seed Oil (variant product): polyunsaturated fats including Omega-3 fatty acids and antioxidant properties from carotenoids, primarily Lutein, tocopherols, and tocotrienols (raspberry seed oil)
Combination 2 Mango Butter (variant product for consumers allergic to latex): Oleic acid, Stearic acid, Palmitic acid, Linoleic acid, Arachidic acid, Vitamin A, Vitamin C, Vitamin E At least one specification heading is required. Please delete this heading section if it is not applicable to your application. For more information regarding the headings of the specification, please see MPEP 608.01(a).

1. A topical composition comprising at least 5 active plant-based ingredients which each have at least one activity that include but are not limited to: moisturizes skin that lasts hours after application, reduces the appearance of scars, provides UV protection without constant reapplication, tightens most skin types, helps with acne prone and blemished skin, reduces redness and skin irritation, battles free radicals, removes make up. This combination also leads to anti-inflammatory, antianxiety, and antimicrobial results after a small to moderate amount is applied the skin that is exposed to the elements. The ingredients that supply these benefits are listed included the proportion of the jar they are mixed in: Shea Butter (50%), Coconut Oil (25%), Grapeseed Oil (5%), Carrot Oil (10%) or Raspberry Oil for SPF of 30-50, and 5% Lavender.

2. The composition of claim 1 where the ingredients are blended using an emulsifier and applied once a day.

3. The composition of claim 1 where Mango Butter is used instead of Shea Butter to make a variant for those with allergies to latex.
